(19) United States
(12) Patent Application Publication
(10) Pub. No.: US 2025/0345767 A1
(43) Pub. Date: Nov. 13, 2025

(54) METHODS AND DEVICES FOR GENERATING CORE-SHELL DROPLETS AND PARTICLES

(71) Applicant: The Trustees of Princeton University, Princeton, NJ (US)

(72) Inventors: Maksym MEZHERICHER, Princeton, NJ (US); Zehao PAN, Plainsboro Township, NJ (US); Howard A. STONE, Princeton, NJ (US)

(73) Assignee: The Trustees of Princeton University, Princeton, NJ (US)

(21) Appl. No.: 18/871,291

(22) PCT Filed: Jun. 6, 2023
(86) PCT No.: PCT/US2023/024520

§ 371 Date: Dec. 3, 2024

Related U.S. Application Data
(60) Provisional application No. 63/349,378, filed on Jun. 6, 2022.
(60) Provisional application No. 63/358,397, filed on Jul. 5, 2022.

(51) Int. Cl. B01J 13/04; B01F 33/3011

(52) U.S. Cl. CPC B01J 13/043; B01F 33/3011

(57) ABSTRACT

Disclosed is a simple and cost-effective technique for generation of high-throughput aerosols of uniform-diameter submillimeter-size core-shell particles. An aerosol may be created by using, e.g., a first tube filled with liquid and having a small hole through a sidewall, then passing a fluid through the liquid via a second tube passing partially though the first tube at a location above the small hole, forming coaxial flow through the small hole. The diameter of generated core-shell particles scales with the inner and outer diameter of the gas tube nozzle, enabling control on the size of the produced particles. Further disclosed is a simple, scalable and cost-effective technique that enables microencapsulation of various materials. Including highly viscous materials, into sub-10 μm particles. A specially designed atomizing tube interacts with bubbles formed in a liquid comprising a plurality of immiscible liquid layers to generate aerosols of droplets which have layered core-shell structure.

### CROSS-REFERENCE TO RELATED APPLICATIONS

The present application claims priority to is drawn to U.S. provisional patent application 63/349,378, filed Jun. 6, 2022, and U.S. provisional patent application 63/358,397, filed Jul. 5, 2022, each of which are incorporated by reference herein in its entirety.

### TECHNICAL FIELD

The present disclosure is drawn to the techniques for forming core-shell droplets and particles.

### BACKGROUND

There are many possible applications of aerosols with core-shell particles, for example direct delivery of materials for medical applications (e.g., delivery to nose, throat); usage of core-shell particles for ultrasonic diagnostics; encapsulation of materials for storage, protection, gradual release; production of foams; additive manufacturing; food production; and recreational usage. However, techniques for forming such aerosols that can meet the requirements for those applications. For example, conventional techniques cannot create substantially uniform droplet or particle sizes, at a high rate of production, with a simple system or device, at an affordable cost.

### BRIEF SUMMARY

The presently disclosed devices, systems, and techniques overcome the deficiencies in the prior art.

In various aspects, a device for generating substantially uniform core-shell particles may be provided. For simplicity, within this application, “particle” is used broadly, and includes solid or semi-solid particles, liquid droplets, etc. Such particles may be dispersed in a gaseous fluid, i.e., as an aerosol. The device may include a first tubular member and at least one second tubular member. The first tubular member may have a first end and a second end. The first tubular member may have a first lumen and at least one second lumen. The first lumen may extend from the first end to the second end. Each second lumen may extend from an external surface of the first tubular member, through a sidewall of the first tubular member, to connect to the first lumen at a location a distance (which may be a predetermined distance) in an axial direction from the either end (such as from the first end). The at least one second tubular member (which may be, e.g., a needle) may have a first end positioned external to the first tubular member and a second end within the first lumen and directed towards one of the at least one second lumen. The at least one second tubular member may extend through the sidewall of the first tubular member. The at least one second tubular member may have an annular cross-sectional shape, defined by an inner diameter and a wall thickness.

In this arrangement, in operation, fluid flowing through the second tubular member will exit the second end of the second tubular member, flow through a fluid in the first tubular member, and pass out through a second lumen. The particle will include a “core” formed from the fluid flowing through the second tubular member, the core being surrounded by a “shell” formed from the fluid flowing through the first tubular member.

The device may include one or more connector(s). Each connector may be operably coupled to the first end of the first tubular member, the second end of the first tubular member, or the first end of one of the at least one second tubular member.

The first tubular member may be composed of an elastic material. The at least one second lumen may open or expand when a pressurized liquid is provided into the first lumen.

The at least one second tubular member may be composed of a rigid material. The at least one second tubular member may have an inner diameter D that is 10 μm<D<1 mm and a wall thickness T that is 10 μm<T<1 mm.

The device may include a single second lumen and a single second tubular member. The device may include a plurality of second lumen and a plurality of second tubular members, each directed towards one of the plurality of second lumen. Preferably, the number of second lumen is equal to the number of second tubular members.

The device may include at least one third tubular member that may have an inner diameter larger than an outer diameter of the first tubular member. The first tubular member and the at least one third tubular member may be concentrically positioned, and configured to generate core-shell particles having multiple shells around a core.

The at least one third tubular member may have at least one third lumen that is positioned such that particles exiting the at least one second lumen will also pass through the at least one third lumen. In this arrangement, the inner-most layers of the shell around the core will be formed from fluid flowing through the first tubular member, and the outer-most layer(s) of the shell will be formed from fluid flowing through the third tubular member(s).

The device may include at least one fourth tubular member having an inner diameter larger than an outer diameter of the at least one second tubular member. The at least one second tubular member and the at least one fourth tubular member may be concentrically positioned, and configured to generate a core comprising multiple materials.

In various aspects, a system may be provided. The system may include a device for generating substantially uniform core-shell particles as disclosed herein. The system may include a first fluid source operably coupled to the first end of the first tubular member, the first fluid source configured to provide a first fluid. The system may include a second fluid source operably coupled to the first end of the at least one second tubular member, the second fluid source configured to provide a second fluid. In some embodiments, the first fluid is a liquid and the second fluid is a gas. In some embodiments, the first fluid and the second fluid are different liquids. In some embodiments, the first fluid and the second fluid are free of surfactants.

The system may include a container to collect core-shell particles travelling in a path extending away from a second lumen. The system may include at least one controller to control a flow of fluids through the device to allow a core-shell particle to be formed and directed out of the at least one second lumen. The system may include a drying means, a photopolymerization means, or a pyrolysis means operably coupled to the controller(s). The drying, photopolymerization, or pyrolysis means may be configured to transform at least one layer of the core-shell particle formed by the device from a liquid to a solid. The core-shell particle may be transformed into (or as) an aerosol. The core-shell particle may be transformed on a surface.

In various aspects, a kit may be provided. The kit may include a device for generating substantially uniform core-shell particles as disclosed herein. The kit may include a drying means and/or a photopolymerization means.

In various aspects, a method for generating substantially uniform layered core-shell particles may be provided. The method may include providing a first fluid to the first lumen of a device for generating substantially uniform core-shell particles as disclosed herein. A pressure of the first fluid may cause the second lumen to open and form a fluid film that spans the open second lumen. The method may include generating substantially uniform core-shell particles by providing a second fluid to the second tubular member. The second tubular member may be configured to direct the second fluid through the fluid film, resulting in a core-shell particles formed having a shell comprising the first fluid surrounding a core comprising the second fluid. The first fluid and the second fluid may be free of surfactants.

The method may include passing at least one additional fluid through at least one additional tubular member concentrically positioned around the second tubular member, creating a single-shell sphere with a multiple-material core of gas, liquid, or combination thereof.

The method may include allowing the core-shell particle to pass through one additional fluid stream passing through at least one tubular member concentrically positioned around the first tubular member, creating a multi-shell sphere around a core of gas or liquid.

The method may include drying the core-shell particle. The method may include photopolymerizing the shell and/or core of the core-shell particle. The method may include pyrolyzing the core-shell particle. The method may include allowing a chemical reaction to occur in at least one layer of the core-shell particle. The method may include collecting the core-shell particle. The method may include allowing the core-shell particles to form a foam.

In various embodiments, each core-shell particles may comprise either (i) a microsphere having a one-layer fluid shell and a one-material fluid core, (ii) a microsphere having a multi-layer fluid shell and a one-material fluid core, (iii) a microsphere having a one-layer fluid shell and a multi-material fluid core, or (iv) a microsphere having a multi-layer fluid shell and a multi-material fluid core.

The fluid shell may comprise a liquid. The fluid shell may comprise a solid. The fluid core may comprise a gas. The fluid core may comprise a liquid. The fluid core may comprise a solid.

The method may include adjusting the pressure of the first fluid to control an outlet area of the second lumen. The method may include adjusting the pressure of the first fluid to control a size of the core-shell particles.

The second tubular member, the first lumen, and the pressure of the first fluid may be configured to provide a core-shell particle having an outer diameter that is about 200 microns or less. At least 10 mL/min of the core-shell particles may pass through a single second lumen.

In various aspects, an alternate system may be provided, the system configured to create micron-size droplets, submicron-size droplets, or particles containing microencapsulated materials. The system may include an atomization (or “aerosolization”) chamber. The system may include a tube within the atomization chamber. The tube may be configured to be partially submerged in a liquid. The tube may include openings through a sidewall of the tube, the openings arranged such that at least some openings are configured to direct a gas jet towards a bubble on a surface of the liquid to form micron-size droplets, submicron-size droplets, or particles containing microencapsulated materials. The liquid may include a plurality of immiscible liquid layers.

The plurality of immiscible liquid layers may include a first layer comprising a first material R, and a second layer comprising a second material G, and a third material B in the first layer and/or the second layer, where R, G, and B are selected such that γ_{RB}>γ_{RG}+γ_{GB}, where γ_{RB} is the interface surface tension between the materials R and B, γ_{RG} is the interface surface tension between the materials R and G, and γ_{GB} is the interface surface tension between the materials G and B.

The micron-size droplets, submicron-size droplets, or particles containing microencapsulated material may include a single-layer shell. The droplets or particles with a single layer shell may include a single-material core. The droplets or particles with a single layer shell may include a multi-material core. The micron-size droplets, submicron-size droplets, or particles containing microencapsulated material comprises a multi-layer shell. The droplets or particles with a multi-layer shell may include a single-material core. The droplets or particles with a multi-layer shell may include a multi-material core. In some embodiments, all shells may be liquid or solid, or one or more shells may be liquid and one or more shells may be solid. In some embodiments, the core may be liquid or solid, or the core may include a mixture of solid and liquid materials.

The system may include a guiding tube coupled to a top portion of the atomization chamber. The guiding tube may be ultraviolet (UV)-transparent. The guiding tube may be configured to have heated, thermo-insulated or cooled walls. The guiding tube may include a bottom portion coupled to the atomization chamber. The bottom portion and/or sidewalls of the guiding tube may be configured to have apertures for entrainment of outside ambient gas to mix with an aerosol in the guiding tube.

The system may include an ultraviolet (UV) light source configured to illuminate an aerosol in the guiding tube. The system may include an electrical heating or cooling coil coupled to the guiding tube. The system may include a parabolic mirror configured to concentrating solar energy irradiating the guiding tube. The system may include a burner coupled to an end of the guiding tube, the burner configured to solidify, dehydrate, or pyrolyze aerosol droplets. The system may include at least one chamber configured to form a dry particle aerosol via solvent evaporation of a submicron droplet aerosol. The system may include a particle collector configured to collect dry particles from a dry particle aerosol. The system may include a liquid, solid or electrostatic filter to capture particulate material from an aerosol stream flowing in the guiding tube.

In various aspects, a method for creating micron-size droplets, submicron-size droplets, or particles containing microencapsulated materials may be provided. The method may include providing a liquid comprising a plurality of immiscible liquid layers. The method may include aerating the liquid in an atomization chamber to form bubbles passing through each of the plurality of immiscible liquid layers, such that the bubbles rise to a surface of the liquid. The method may include forming a submicron droplet aerosol by causing a gas jet to be directed through an opening in a tube towards at least one of the bubbles in the atomization chamber.

The method may include heating or cooling a guiding tube coupled to a top portion of the atomization chamber. The method may include entraining outside ambient gas through apertures in a portion of the guiding tube coupled to the atomization chamber and/or sidewalls of the guiding tube to mix with an aerosol in the guiding tube. The method may include photopolymerizing a material in a bubble by directing ultraviolet (UV) light towards an aerosol in the guiding tube. The method may include solidifying, dehydrating, or pyrolyzing aerosol droplets. The method may include forming a dry particle aerosol via solvent evaporation of the submicron droplet aerosol. The method may include forming a powder of submicron or nano-structured particles by passing the dry particle aerosol through a particle collector.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a cross-section of an embodiment of a device.

FIG. 2 is an illustration showing a perspective view of an embodiments of a first tubular member.

FIG. 3 is an illustration showing a perspective view of an embodiment of a second tubular member.

FIG. 4 is an illustration of a cross-section of an embodiment of a device with two second tubular members.

FIG. 5 is an illustration of a cross-section of an embodiment of a device with a third tubular member.

FIG. 6 is an illustration of a cross-section of an embodiment of a device with a fourth tubular member.

FIG. 7 is an illustration of an embodiment of a system.

FIG. 8 is a flowchart of an embodiment of a method.

FIG. 9 is an illustration of an embodiment of a drug delivery device incorporating a particle-generating device as disclosed herein.

FIG. 10 is a schematic diagram of an exemplary setup for atomization of multi-layer particles.

FIG. 11A is a graphical illustration of one embodiment of a method for aerosol particle production.

FIG. 11B is a graphical illustration of an embodiment of a perforated tube or pipe within an atomization chamber.

FIG. 12 is a flowchart of an embodiment of a method.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

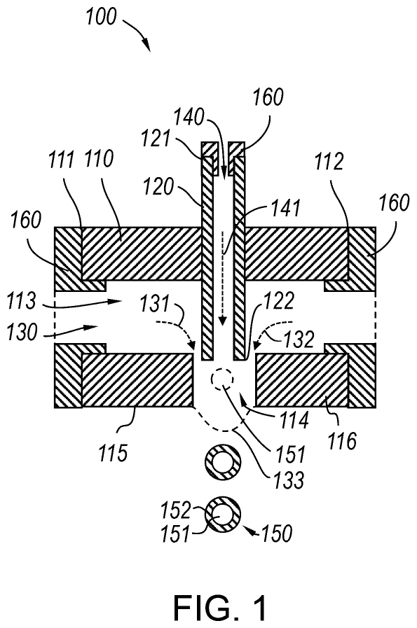

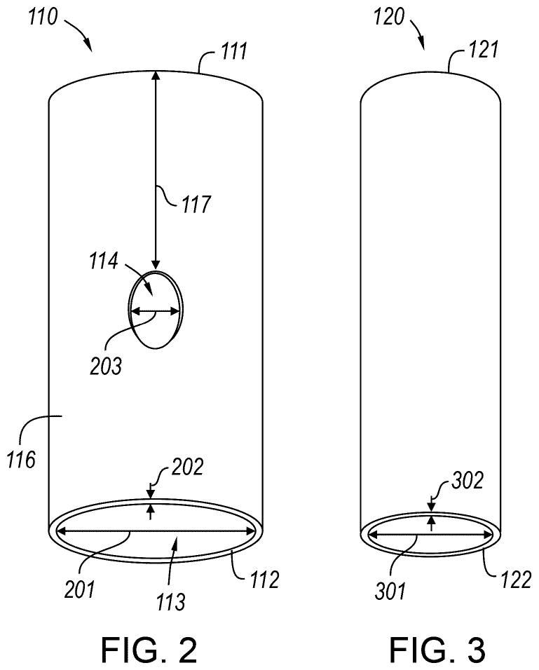

In various aspects, a device for generating substantially uniform core-shell particles may be provided. Referring to FIGS. 1-3, a device 100 may include a first tubular member 110 and at least one second tubular member 120.

The first tubular member 110 may have a first end 111 and a second end 112 axially separated from the first end. The first tubular member may have a first lumen 113 and at least one second lumen 114. The first lumen 113 may extend from the first end to the second end. Each second lumen 114 may extend from an external surface 115 of the first tubular member 110, through a sidewall 116 of the first tubular member 110, to connect to the first lumen 113 at a location a distance 117 (which may be a predetermined distance) in an axial direction from the either end (such as from the first end).

The first tubular member 110 may have a first fluid 130 flowing within it. In some embodiments, some or all of the first fluid 130 may flow from the first end towards the second end. In some embodiments, the some or all of the first fluid 130 may flow from the second end towards the first end. In some embodiments, fluid flowing from the first end may flow 131 around the second tubular member 120 and may enter the second lumen 114. In some embodiments, fluid flowing from the first end may flow 132 around the second tubular member 120 and may enter the second lumen 114. The first fluid 130 entering the second lumen 114 may form a surface 133 that, at the second lumen 114, extends radially outward from the external surface 115 of the first tubular member 110.

The first tubular member 110 may be composed of a rigid material. For example, the tubular member may be composed of polyvinyl chloride or stainless steel.

The first tubular member 110 may be composed of an elastic or expandable material. For example, the tubular member may be composed of latex or natural rubber. The at least one second lumen 114 may open or expand when a pressurized liquid is provided into the first lumen 113.

The first tubular member 110 may be composed of a polymer, such as a polyethylene, a polypropylene, or a polyurethane.

The first tubular member 110 may have an inner diameter (D1) 201, defined by the first lumen 113, that is 10 μm<D1<10 mm and a wall thickness (T1) 202 that is 10 μm <T1<5 mm. In some embodiments, D1 may be less than or equal to 50 mm. In some embodiments, D1 may be less than or equal to 40 mm. In some embodiments, D1 may be less than or equal to 30 mm. In some embodiments, D1 may be less than or equal to 20 mm. In some embodiments, D1 may be less than or equal to 10 mm. In some embodiments, D1 may be less than or equal to 5 mm. In some embodiments, D1 may be less than or equal to 3 mm. In some embodiments, T1 may be less than or equal to 10 mm. In some embodiments, T1 may be less than or equal to 5 mm. In some embodiments, T1 may be less than or equal to 3 mm. In some embodiments, T1 may be less than or equal to 2 mm. In some embodiments, T1 may be less than or equal to 1 mm.

Note, as used herein, the term “diameter” is intended to refer to the largest separation within the lumen between two opposing surfaces through which a fluid of interest flows. For lumen of a circular cross-section, this is the diameter lumen. For lumen of a rectangular cross-section (for example), this could be either a length or width of the rectangle, whichever is greater.

The cross-section of the lumen may vary. In some embodiments, the first lumen 113 may have a circular cross-section. In some embodiments, the first lumen 113 may have a rectangular cross-section. In some embodiments, the first lumen 113 may have an oval cross-section.

The at least one second lumen 114 may have an inner diameter (D2) 203 that is 20 μm<D2<5 mm. In some embodiments, D2 may be less than or equal to 5 mm. In some embodiments, D2 may be less than or equal to 4 mm. In some embodiments, D2 may be less than or equal to 3 mm. D2 may be less than or equal to 4 mm. In some embodiments, D2 may be less than or equal to 2 mm.

The at least one second tubular member 120 (which may be, e.g., a needle) may have a first end 121 positioned or disposed external to the first tubular member 110 and a second end 122 position or disposed within the first lumen 113 and directed towards one of the at least one second lumen 114. Said differently, the second end must be configured such that a second fluid 140 passing through the second tubular member will then pass through the second lumen 114 the second end is directed towards. The at least one second tubular member 120 may extend through the sidewall of the first tubular member 110. The at least one second tubular member 120 may have an annular cross-sectional shape, defined by an inner diameter and a wall thickness.

In this arrangement, in operation, a second fluid 140 flowing 141 through the second tubular member 120 will exit the second end 122 of the second tubular member 120, forming a base “core” 151 flowing through first fluid 130 in the second lumen 114 of the first tubular member 110, then pass out through the second lumen 114, forming discrete particles 150 containing the core 151 and an outer “shell” 152 formed from the first fluid 130.

The at least one second tubular member 120 may be composed of a rigid material. The rigid material may be, e.g., stainless steel, glass, etc. As used herein, the term “rigid” refers to a component having a substantially stiff structure that resists bending and is not generally flexible.

The at least one second tubular member 120 may have an inner diameter (D) 301 that is 10 μm<D<1 mm and a wall thickness (T) 302 that is 10 μm<T<1 mm. In some embodiments, the second lumen 114 may have an inner diameter (D2) that is at least 1 mm larger than the inner diameter D of the second tubular member 120. In some embodiments, D2 may be at least 2 mm larger than the inner diameter D of the second tubular member 120. In some embodiments, D2 may be at least twice as large as the inner diameter D of the second tubular member 120.

Because the disclosed process involves a liquid flow through a hole in an elastic tube, the viscosity of liquid is one of the limiting factors. Another limit is liquid pressure that can sustain elastic tube material without rupture. Also, the diameter of the generated particles depends at least partially on the inner and outer diameters of the second tubular member 120, which limits the smallest achievable diameter of the generated particles. In particular, due to the latter limitation, one tested prototype using 34 gage steel blunt needles, with an 83 μm inner diameter and a 184 μm outer diameter, obtained 200 μm as the smallest diameter of generated uniform-size core-shell particles. Consequently, the device may require a second tubular member 120 with an outer diameter smaller than the desired target diameter of the core-shell particles. In some embodiments, the outer diameter may be 80-99% of the target diameter of the core-shell particle. In some embodiments, the outer diameter may be 85-97% of the target diameter of the core-shell particle. In some embodiments, the outer diameter may be 90-95% of the target diameter of the core-shell particle.

In some embodiments, the various fluids used to form the droplets, particles, etc., comprise, consist, or consists essentially of, one or more solvents. In some embodiments, the various fluids used to form the droplets, particles, etc., comprise, consist, or consists essentially of, one or more dispersed active chemical or biological materials.

### Active Chemical or Biological Material

The active chemical or biological material can be any appropriate material as understood by those of skill in the art, depending on the purpose of the aerosol.

The active chemical may include, for example, nutraceuticals, pharmaceuticals, and/or supplements.

For example, any drug, therapeutically acceptable drug salt, drug derivative, drug analog, drug homologue, or polymorph can be used in the present invention. Suitable drugs for use with the present invention can be found in the Physician's Desk Reference, 71^{st} Edition, the content of which is hereby incorporated by reference.

In certain embodiments, psychoactive drugs and analgesics, including but not limited to opioids, opiates, stimulants, tranquilizers, sedatives, anxiolytics, narcotics and drugs that can cause psychological and/or physical dependence can be used. In one embodiment, the drug for use in the present invention can include amphetamines, amphetamine-like compounds, benzodiazepines, and methyl phenidate or combinations thereof. In another embodiment, drugs may include any of the resolved isomers of the drugs described herein, and/or salts thereof.

Other non-limiting drugs that may be used include alfentanil, amphetamines, buprenorphine, butorphanol, carfentanil, codeine, dezocine, diacetylmorphine, dihydrocodeine, dihydromorphine, diphenoxylate, diprenorphine, etorphine, fentanyl, hydrocodone, hydromorphone, β-hydroxy-3-methylfentanyl, levo-α-acetylmethadol, levorphanol, lofentanil, meperidine, methadone, methylphenidate, morphine, nalbuphine, nalmefene, oxycodone, oxymorphone, pentazocine, pethidine, propoxyphene, remifentanil, sufentanil, tilidine, and tramodol, salts, derivatives, analogs, homologues, polymorphs thereof, and mixtures of any of the foregoing.

Further non-limiting examples of chemicals that may be utilized include dextromethorphan (3-Methoxy-17-methy-9a,13a,14a-morphinan hydrobromide monohydrate), N-{1-[2-(4-ethyl-5-oxo-2-tetrazolin-1-yl)-ethyl]-4-methoxymethyl-4-piperidyl} propionanilide (alfentanil), 5,5-diallyl barbituric acid (allobarbital), allylprodine, alpha-prodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methyl phenethylamine (amphetamine), 2-(α-methylphenethyl-amino)-2-phenyl acetonitrile (amphetaminil), 5-ethyl-5-isopentyl barbituric acid (amobarbital), anileridine, apocodeine, 5,5-diethyl barbituric acid (barbital), benzylmorphine, bezitramide, 7-bromo-5-(2-pyridyl)-1H-1,4-benzodiazepin-2(3H)-one (bromazepam), 2-bromo-4-(2-chlorophenyl)-9-methyl-6H-thieno[3,2-f][1,2,4]-triazolo[4,3-a][1,4]diazepine (brotizolam), 17-cyclopropylmethyl-4,5α-epoxy-7α[(S)-1-hydroxy-1,2,2-trimethylpropyl]-6-methoxy-6,14-endo-ethanomorphinan-3-ol (buprenorphine), 5-butyl-5-ethyl barbituric acid (butobarbital), butorphanol, (7-chloro-1,3-dihydro-1-methyl-2-oxo-5-phenyl-2H-1,4-benzodiazepin-3-yl)-dimethyl carbamate (camazepam), (1S,2S)-2-amino-1-phenyl-1-propanol (cathine/D-norpseudoephedrine), 7-chloro-N-methyl-5-phenyl-3H-1,4-benzodiazepin-2-ylamine-4 oxide (chlordiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepin-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]-diazepin-2(3H)-one (clotiazepam), 10-chloro-11b-(2-chlorophenyl)-2,3,7,11b-tetrahydrooxazolo[3,2-d][1,4]benzodiazepin-6(5H)-one (cloxazolam), (−)-methyl-[3β-benzoyloxy-2β(1αH,5αH)-tropane carboxylate (cocaine), 4,5α-epoxy-3-methoxy-17-methyl-7-morphinen-6α-ol (codeine), 5-(1-cyclohexenyl)-5-ethyl barbituric acid (cyclobarbital), cyclorphan, cyprenorphine, 7-chloro-5-(2-chlorophenyl)-1H-1,4-benzodiazepin-2(3H)-one (delorazepam), desomorphine, dextromoramide, (+)-(1-benzyl-3-dimethylamino-2-methyl-1-phenylpropyl) propionate (dextropropoxyphene), dezocine, diampromide, diamorphone, 7-chloro-1-methyl-5-phenyl-1H-1,4-benzodiazepin-2(3H)-one (diazepam), 4,5α-epoxy-3-methoxy-17-methyl-6α-morphinanol (dihydrocodeine), 4,5α-epoxy-17-methyl-3,6α-morphinandiol(dihydromorphine), dimenoxadol, dimethyl thiambutene, dioxaphetyl butyrate, dipipanone, (6aR,10aR)-6,6,9-trimethyl-3-pentyl-6a,7,8,10a-tetrahydro-6H-benzo[c]chromen-1-ol (dronabinol), eptazocine, 8-chloro-6-phenyl-4H-[1,2,4]triazolo[4,3-a][1,4]benzodiazepine (estazolam), ethoheptazine, ethyl methyl thiambutene, ethyl-[7-chloro-5-(2-fluorophenyl)-2,3-dihydro-2-oxo-1H-1,4-benzodiazepin-3-carboxylate] (ethyl loflazepate), 4,5α-epoxy-3-ethoxy-17-methyl-7-morphinen-6α-ol (ethylmorphine), etonitrazene, 4,5α-epoxy-7α-(1-hydroxy-1-methylbutyl)-6-methoxy-17-methyl-6,14-endo-etheno-morphinan-3-ol (etorphine), N-ethyl-3-phenyl-8,9,10-trinorboman-2-ylamine (fencamfamine), 7-[2-(α-methylphenethylamino)-ethyl]theophylline (fenethylline), 3-(α-methylphenethylamino) propionitrile (fenproporex), N-(1-phenethyl-4-piperidyl) propionanilide (fentanyl), 7-chloro-5-(2-fluorophenyl)-1-methyl-1H-1,4-benzodiazepin-2(3H)-one (fludiazepam), 5-(2-fluorophenyl)-1-methyl-7-nitro-1H-1,4-benzodiazepin-2-(3H)-one (flunitrazepam), 7-chloro-1-(2-diethylaminoethyl)-5-(2-fluorophenyl)-1H-1,4-benzodiazepin-2(3H)-one (flurazepam), 7-chloro-5-phenyl-1-(2,2,2-trifluoroethyl)-1H-1,4-benzodiazepin-2(3H)-one (halazepam), 10-bromo-11b-(2-fluorophenyl)-2,3,7,11b-tetrahydro[1,3]oxazolo[3,2-d][1,4]benzodiazepin-6(5H)-one (haloxazolam), heroin, 4,5α-epoxy-3-methoxy-17-methyl-6-morphinanone (hydrocodone), 4,5α-epoxy-3-hydroxy-17-methyl-6-morphinanone (hydromorphone), hydroxypethidine, isomethadone, hydroxymethyl morphinan, 11-chloro-8,12b-dihydro-2,8-dimethyl-12b-phenyl-4H-[1,3]oxazino[3,2-d][1,4]benzodiazepin-4,7(6H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenyl-3-heptanone (levomethadone), (−)-17-methyl-3-morphinanol (levorphanol), levophenacyl morphan, lofentanil, 6-(2-chlorophenyl)-2-(4-methyl-1-piperazinylmethylene)-8-nitro-2H-imidazo[1,2a][1,4]benzodiazepin-1(4H)- one (loprazolam), 7-chloro-5-(2-chlorophenyl)-3-hydroxy-1H-1,4-benzodiazepin-2(3H)-one (lorazepam), 7-chloro-5-(2-chlorophenyl)-3-hydroxy-1-methyl-1H-1,4-benzodiazepin-2(3H)-one (lormetazepam), 5-(4-chlorophenyl)-2,5-dihydro-3H-imidazo[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenetylamine (mefenorex), meperidine, 2-methyl-2-propyl trimethylene dicarbamate (meprobamate), meptazinol, metazocine, methylmorphine, N,α-dimethylphenethylamine (methamphetamine), (±)-6-dimethylamino-4,4-diphenyl-3-heptanone (methadone), 2-methyl-3-o-tolyl-4(3H)-quinazolinone (methaqualone), methyl-[2-phenyl-2-(2-piperidyl)acetate] (methyl phenidate), 5-ethyl-1-methyl-5-phenyl barbituric acid (methyl phenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl) acetamide (modafinil), 4,5α-epoxy-17-methyl-7-morphinene-3,6α-diol (morphine), myrophine, (±)-trans-3-(1,1-dimethylheptyl)-7,8,10,10α-tetrahydro-1-hydroxy-6,6-dimethyl-6H-dibenzo[b,d]pyran-9(6αH)-one (nabilone), nalbuphen, nalorphine, narceine, nicomorphine, 1-methyl-7-nitro-5-phenyl-1H-1,4-benzodiazepin-2(3H)-one (nimetazepam), 7-nitro-5-phenyl-1H-1,4-benzodiazepin-2(3H)-one (nitrazepam), 7-chloro-5-phenyl-1H-1,4-benzodiazepin-2-(3H)-one (nordazepam), norlevorphanol, 6-dimethylamino-4,4-diphenyl-3-hexanone (normethadone), normorphine, norpipanone, the coagulated juice of the plants belonging to the species Papaver somniferum (opium), 7-chloro-3-hydroxy-5-phenyl-1H-1,4-benzodiazepin-2-(3H)-one (oxazepam), (cis-trans)-10-chloro-2,3,7,11b-tetrahydro-2-methyl-11b-phenyloxazolo[3,2- d][1,4]benzodiazepin-6-(5H)-one (oxazolam), 4,5α-epoxy-14-hydroxy-3-methoxy-17-methyl-6-morphinanone (oxycodone), oxymorphone, plants and plant parts of the plants belonging to the species Papaver somniferum (including the subspecies setigerum) (Papaver somniferum), papaveretum, 2-imino-5-phenyl-4-oxazolidinone (pernoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl) barbituric acid (pentobarbital), ethyl-(1-methyl-4-phenyl-4-piperidine-carboxylate) (pethidine), phenadoxone, phenomorphan, phenazocine, phenoperidine, piminodine, pholcodeine, 3-methyl-2-phenyl morpholine (phenmetrazine), 5-ethyl-5-phenyl barbituric acid (phenobarbital), α,α-dimethyl phenethylamine (phentermine), 7-chloro-5-phenyl-1-(2-propinyl)-1H-1,4-benzodiazepin-2(3)-one (pinazepam), α-(2-piperidyl)benzhydryl alcohol (pipradol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepin-2(3H)-one (prazepam), profadol, proheptazine, promedol, properidine, propoxyphene, N-(1-methyl-2-piperidinoethyl)-N-(2-pyridyl) propionamide, methyl-{3-[4-methoxycarbonyl-4-(N-phenylpropaneamido)piperidino]propanoate} (remifentanil), 5-sec.-butyl-5-ethyl barbituric acid (secbutabarbital), 5-allyl-5-(1-methylbutyl) barbituric acid (secobarbital), N-{4-methoxymethyl-1-[2-(2-thienypethyl]-4-piperidyl}propionanilide (sufentanil), 7-chloro-2-hydroxy-methyl-5-phenyl-1H-1,4-benzodiazepin-2-(3H)-one (temazepam), 7-chloro-5-(1-cyclohexenyl)-1-methyl-1H-1,4-benzodiazepin-2(3H)-one (tetrazepam), ethyl-(2-dimethylamino-1-phenyl-3-cyclohexane-1-carboxylate) (tilidine (cis and trans)), tramadol, 8-chloro-6-(2-chlorophenyl)-1-methyl-4H-[1,2,4]triazolo[4,3-a][1,4]benzodiazepine (triazolam), 5-(1-methylbutyl)-5-vinyl barbituric acid (vinylbital), (1R*,2R*)-3-(3-dimethylamino-1-ethyl-2-methyl-propyl) phenol, (1R,2R,4S)-2-[dimethylamino)methyl-4-(p-fluorobenzyloxy)-1-(m-methoxyphenyl) cyclohexanol, each optionally in the form of corresponding stereoisomeric compounds as well as corresponding derivatives, especially esters or ethers, and all being physiologically compatible compounds, especially salts and solvates.

In some embodiment, the method utilizes one or more opioids such as hydrocodone, hydromorphone, morphine and oxycodone and/or salts thereof.

Non-limiting examples of an API that may be utilized include inorganic synthetic drugs (such as Aluminum hydroxide, magnesium trisilicate, etc.), or organic synthetic drugs (such as aspirin, chloramphenicol, caffeine, etc.). APIs may also include antibiotics (such as Aminoglycosides such as Amikacin, Gentamicin, Kanamycin, etc.), Ansamycins (such as Geldanamycin, Herbimycin, etc.), Carbapenems (sch as Ertapenem, Doripenem, Cilastatin, etc.), Cephalosporins (including 1^{st}, 2^{nd}, 3^{rd}, 4^{th}, and/or 5^{th} generation Cephalosporins such as Cefadroxil, Cefazolin, Caphradine, Cefaclor, Cefoxitin, Cefonicid, Cefixime, Cefdinir, Cefdotaxime, Cefepime, Ceftaroline fosamil, Ceftobiprole, etc.), Glycopeptides (such as Teicoplanin, Vancomycin, etc.), Licosamides (such as Clindamycin, etc.), Lipopetides (such as Daptomycin), Mecrolides (such as Azithromycin, Clarithromycin, Fidaxomicin, etc.), Monobactams (such as Aztreonam), Nitrofurans (such as Furazolidone), Oxazolidinones (such as Linezolid, etc.), Penicillins (such as Amoxicillin, etc.), Polypeptides (such as Bacitracin, Colistin, Polymyxin B, etc.), Quinolones/Fluoroquinolones (such as Ciprofloxacin, Enoxacin, Levofloxacin, etc.), Sulfonamides (such as Mafenide, Sulfacetamide, etc.), and Tetracyclines (such as Demeclocycline, Doxycycline, etc.). APIs may also include various phytochemicals or phytochemal containing compounds, including phytoestrogens such as genistein and daidzein, such as isoflavones (e.g., soy isoflavones), flavonoids, phytoalexins (e.g., resveratrol (3,5,4′-trihydroxystilbene)), red clover extract, and phytosterols.

Other active chemicals can include, e.g., essential fatty acids, including polyunsaturated fatty acids, such as omega-3 fatty acids, omega-6 fatty acids, omega-9 fatty acids, conjugated fatty acids, and other fatty acids; oil soluble vitamins, including vitamin D3 and vitamin A palmitate; alpha lipoic acid; other oils; coenzymes, including Coenzyme Q10; and carotenoids, including lycopene, lutein, and zeaxanthin.

Other active chemicals can include therapeutic compounds in various therapeutic oils or plant extracts, including but not limited to cannabinoids, such as cannabidiol. In some embodiments, cannabis oil is utilized.

Other active chemicals can include inorganic materials, including graphene or graphene oxide, and metal oxides such as aluminum oxide, calcium oxide, chromium oxide, cobalt oxide, iron oxide, lead oxide, lithium oxide, silicon dioxide, titanium dioxide, and/or zinc oxide.

Other active chemicals can include industrially useful organic materials, including alkanes and unsaturated hydrocarbons.

Other active chemicals can include foods or food additives, including, e.g., NaCl.

In addition to active chemicals, any appropriate biological material may be utilized as well. For example, in some embodiments, the biological material is a biomolecule. That is, a compound comprising of one or more chemical moieties typically synthesized in living organisms. Non-limiting examples of biomolecules include amino acids, nucleotides, polysaccharides or simple sugars, lipids, or a combination thereof.

In some embodiments, the biological material comprises a cell and/or cell debris, in contrast to a purified biomolecule (e.g., a purified enzyme). In some embodiments, the biological material may be, or may be obtained from, viruses (e.g., bacteriophages), cells (e.g., microorganisms), tissues, and organisms (e.g., plants) using conventional, known techniques.

### Solvent

The liquid will generally contain at least one solvent. A preferred embodiment utilizes water as a solvent, but other solvents may be included. In some embodiments, the solvent is a pharmaceutically acceptable solvent. Non-limiting examples of pharmaceutically acceptable solvents include ketones such as acetone, alcohols such as methanol, ethanol, or propanol, a mixture thereof, and a mixed solvent of water with one or more of these solvents. These pharmaceutically acceptable solvents may be used alone or as an appropriate combination of two or more thereof.

In some embodiments, the liquid is non-aqueous. In some embodiments, the solvent is an oil fit for human consumption, such as castor oil, soybean oil, sunflower oil, coconut oil, hemp oil or olive oil. In some embodiments, the solvent comprises, consists essentially of, or consists of one or more saturated fatty acids, one or more unsaturated fatty acids, or a combination thereof.

The concentration of the active chemical or biological material that is present in the liquid is not particularly limited. Preferably, the active chemical or biological material can be dispersed in the liquid. In some embodiments, the concentration of the active chemical or biological material is between 0.01% and 99% by weight of the liquid. In some embodiments, the concentration of the active chemical or biological material is between 0.01% and 50% by weight of the liquid. In some embodiments, the concentration of the active chemical or biological material is between 0.1% and 30% by weight of the liquid. In some embodiments, the concentration of the active chemical or biological material is between 1% and 20% by weight of the liquid.

The device may include one or more connectors 160. Each connector may be operably coupled to the first end 111 of the first tubular member 110, the second end 112 of the first tubular member 110, or the first end 121 of one of the at least one second tubular member 120.

The connectors generally allow the device to be operably coupled to one or more fluid sources, and as such, the connectors may include any appropriate connection to the tubular members—threads, ridges, adhesive, welds, etc. In some embodiments, the connectors are removably coupled to the device. In some embodiments, the connectors are affixed permanently to the device.

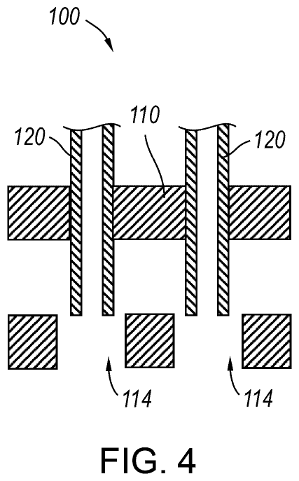

As seen in FIG. 1, the device may include a single second lumen 114 and a single second tubular member 120. Referring to FIG. 4, the device may include a plurality of second lumen 114 and a plurality of second tubular members 120, each directed towards one of the plurality of second lumen 114. Preferably, the number of second lumen 114 is equal to the number of second tubular members 120.

In some embodiments, the plurality of second lumen 114 may be arranged in a linear fashion, separated by an axial distance. In some embodiments, the plurality of second lumen 114 may be separated by an axial distance and a circumferential distance. For example, if the first lumen 113 extends from left to right, a second lumen 114 may be oriented directly downward (e.g., vertically), while an adjacent second lumen 114 may be separated a few centimeters to the left or right and may be oriented in a direction, e.g., up to 30, 60, 90, or 180 degrees from vertically downward.

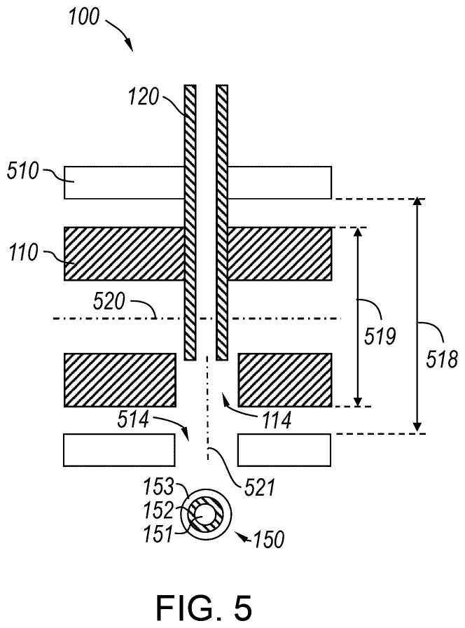

Referring to FIG. 5, the device may include at least one third tubular member 510 that may have an inner diameter 518 larger than an outer diameter 519 of the first tubular member 110. The first tubular member 110 and the at least one third tubular member 510 may be concentrically positioned (e.g., around a central axis 520 of the first tubular member 110), and configured to generate core-shell particles 150 having multiple shells 152, 153 around a core 151. The outer shell(s) are from fluids flowing through the third tubular member(s) 510.

The at least one third tubular member 510 may have at least one third lumen 514 that is positioned such that particles exiting the at least one second lumen 114 will also pass through the at least one third lumen 514. The second lumen 114 and the third lumen 514 may coaxial (e.g., central axis 521 of the second lumen 114 and the third lumen 514 may be the same).

In these arrangements, the inner-most layers of the shell around the core will be formed from fluid flowing through the first tubular member 110, and the outer-most layer(s) of the shell will be formed from fluid flowing through the third tubular member(s) 510.

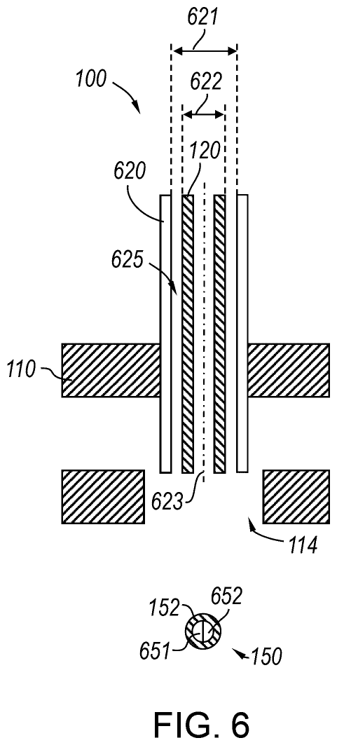

Referring to FIG. 6, the device may include at least one fourth tubular member 620 having an inner diameter 621 larger than an outer diameter 622 of the at least one second tubular member 120. The at least one second tubular member 120 and the at least one fourth tubular member 620 may be concentrically positioned (e.g., around central axis 623). The tubular members may be configured to generate a core comprising multiple materials 651, 652. Note, for ease of illustration, the particle 150 in FIG. 6 is shown as having a core with a left half being one material and the right have being a second material. However, it is understood that any arrangement of the materials can be produced, depending on operating conditions, fluid selection, etc. In some embodiments, the materials forming the core are homogenously distributed. In some embodiments, the materials forming the core are heterogeneously distributed. In some embodiments, the materials forming the core are arranged in a layered fashion.

As will be understood, combinations of these configurations may be used. For example, in one embodiment, the device includes a single first tubular member 110, a single second tubular member 120, a single third tubular member 510, and a singular fourth tubular member 620. In some embodiments, a single second and fourth tubular member 620 may form an injection device, and there may be two or more injection devices introduced into the same first tubular member 110 (or first tubular member 110 and third tubular member(s) 510).

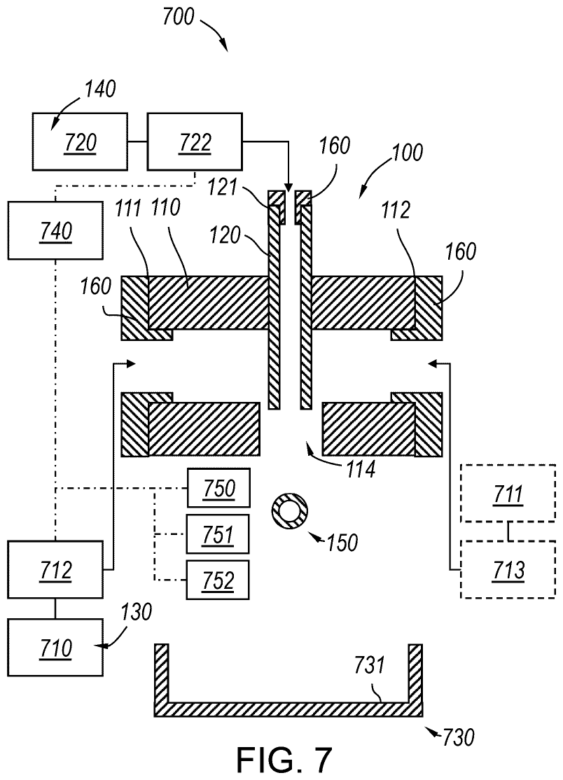

In various aspects, a system may be provided. Referring to FIG. 7, a system 700 may include a device 100 for generating substantially uniform core-shell particles as disclosed herein. The system may include a first fluid source 710 operably coupled to the first end 111 of the first tubular member 110, the first fluid source 710 configured to provide a first fluid 130. The system may include a pump 712 or other means of providing and controlling the flow of fluid to the first tubular member 110. As will be understood, the pump 712 may additionally, or alternatively, provide the first fluid 130 to the second end. Optionally, an additional pump 713 and/or fluid source 711 could be used to provide the first fluid 130 to the second end, if desired.

The system may include a second fluid source 720 operably coupled to the first end 121 of the at least one second tubular member 120, the second fluid source 720 configured to provide a second fluid 140.

The first fluid 130 may include a liquid. The first fluid 130 may include, e.g., one or more UV-curable resins. The first fluid 130 may include one or more volatile solvents. The first fluid 130 may include one or more solids, such as a particulate. The first fluid 130 may include a colorant, such as a pigment or dye.

The second fluid 140 may include a liquid. The second fluid 140 may include a gas. The second fluid 140 may include a particulate. The second fluid 140 may include one or more amino acids. The second fluid 140 may include one or more pharmaceutically active ingredients. In some embodiments, the first fluid 130 and the second fluid 140 are free of surfactants.

The system may include a container 730 to collect core-shell particles 150 travelling in a path extending away from a second lumen 114.

The system may include at least one controller 740 that, collectively, controls a flow of fluids through the device to allow a core-shell particle to be formed and directed out of the at least one second lumen 114. The controller may be connected to, e.g., one or more components, such as pumps 712, 722.

The term “controller” is intended to include any analog or digital means for controlling a process, and may include one or more circuits, and/or one or more processors.

The term “processor” is used herein to include, but not limited to, any integrated circuit or any other electronic device (or collection of electronic devices) capable of performing an operation on at least one instruction, including, without limitation, a microprocessor (μP), a microcontroller (μC), a Digital Signal Processor (DSP), or any combination thereof. A processor may further be a Reduced Instruction Set Core (RISC) processor, a Complex Instruction Set Computing (CISC) microprocessor, a Microcontroller Unit (MCU), or a CISC-based Central Processing Unit (CPU). The hardware of the processor may be integrated onto a single substrate (e.g., silicon “die”), or distributed among two or more substrates. Furthermore, various functional aspects of the processor may be implemented solely as a software (or firmware) associated with the processor. The term “circuit” may refer to one or more passive and/or active components that are arranged to cooperate with one another to provide a desired function.

The system may include a drying means 750 (such as air dryer, freeze dryer, a drum dryer, a tray dryer, etc.), a photopolymerization means 751 (such as a UV, visual light, or IR light source adapted to activate a photopolymer present in one of the fluids), or a pyrolysis means 752 (such as a pyrolysis reactor) operably coupled to/controlled by the controller(s). The drying, photopolymerization, or pyrolysis means may be configured to transform at least one layer of the core-shell particle formed by the device from a liquid to a solid.

The core-shell particle may be transformed into (or as) an aerosol. The core-shell particle may be transformed on a surface (such as a surface 731 of the container 730)

In various aspects, a kit may be provided. The kit may include a device for generating substantially uniform core-shell particles as disclosed herein. The kit may include a drying means and/or a photopolymerization means.

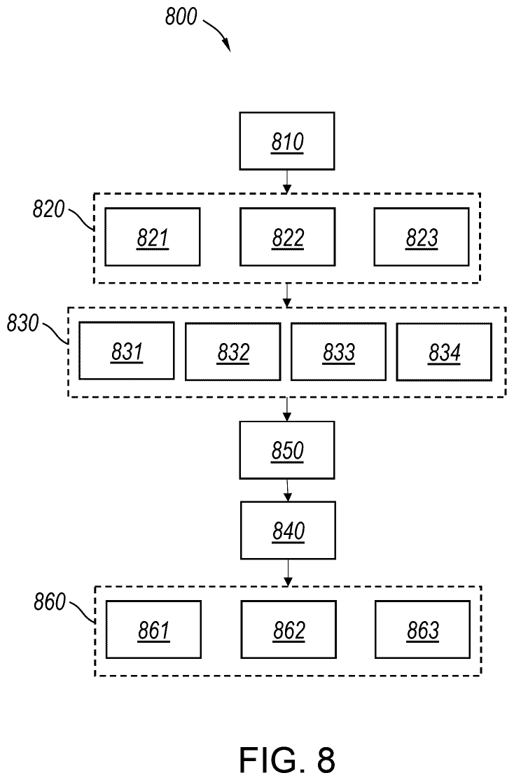

In various aspects, a method for generating substantially uniform layered core-shell particles may be provided. Referring to FIG. 8, the method 800 may include providing 810 a first fluid 130 to the first lumen 113 of a device for generating substantially uniform core-shell particles as disclosed herein.

In various embodiments, a pressure of the first fluid 130 may be between 5 kPa and 1000 kPa gauge pressure.

A pressure of the first fluid 130 may cause the second lumen 114 to open and form a fluid film that spans the open second lumen 114.

The method may include generating 820 substantially uniform core-shell particles. This may include providing 821 a second fluid 140 to the second tubular member 120. The second tubular member 120 may be configured to direct the second fluid 140 through the fluid film, resulting in a core-shell particles formed having a shell comprising the first fluid 130 surrounding a core comprising the second fluid 140. The first fluid 130 and the second fluid 140 may be free of surfactants.

In various embodiments, a pressure of the second fluid 140 may be between 5 kPa and 2000 kPa gauge pressure.

Generating particles may include passing 822 at least one additional core fluid through at least one additional tubular member concentrically positioned around the second tubular member 120, creating a single-shell sphere with a multiple-material core of gas, liquid, or combination thereof. The additional core fluid may be provided at the same pressure as the second fluid 140. The additional core fluid may be provided at a different pressure as the second fluid 140. In various embodiments, a pressure of the additional core fluid may be between 5 kPa and 2000 kPa gauge pressure.

Generating particles may include allowing 823 the core-shell particle to pass through one additional fluid stream (“shell fluid”) passing through at least one tubular member concentrically positioned around the first tubular member 110, creating a multi-shell sphere around a core of gas or liquid. The additional shell fluid may be provided at the same pressure as the first fluid 130. The additional shell fluid may be provided at a different pressure as the first fluid 130. In various embodiments, a pressure of the additional shell fluid may be between 5 kPa and 1000 kPa gauge pressure.

The method may include modifying 830 the output of from the device in some fashion. The method may include drying 831, or at least partially drying, the core-shell particle (for example, by exposing the particles to hot dry air). The method may include photopolymerizing 832 the shell and/or core of the core-shell particle (for example, by exposing particles containing a photopolymer to a UV light source). The method may include pyrolyzing 833 the core-shell particle (for example, in a pyrolizer). The method may include allowing 834 a chemical reaction to occur in at least one layer of the core-shell particle.

The method may include collecting 840 the core-shell particle. This may include collecting in a container (such as a jar) or a plate.

The method may include allowing 850 the core-shell particles to form a foam. This may occur naturally as the particles are collected. However, inclusion of antifoaming or defoaming agents may prevent the foams from forming.

In various embodiments, each core-shell particles may comprise either (i) a microsphere having a one-layer fluid shell and a one-material fluid core, (ii) a microsphere having a multi-layer fluid shell and a one-material fluid core, (iii) a microsphere having a one-layer fluid shell and a multi-material fluid core, or (iv) a microsphere having a multi-layer fluid shell and a multi-material fluid core.

The fluid shell may comprise a liquid. The fluid shell may comprise a solid. The fluid core may comprise a gas. The fluid core may comprise a liquid. The fluid core may comprise a solid.

In one set of examples, the first fluid 130 was either (i) water, (ii) water mixed with glycerol and polysorbate 80, and the second fluid 140 was air.

The method may include controlling 860 the generation of particles. This may include adjusting 861 the pressure of the first fluid 130 to control an outlet area of the second lumen 114. If the first tubular member 110 is flexible/elastic, increasing pressure can allow the size of the outlet area to increase.

This may include adjusting 862 the pressure, density, surface tension, and viscosity of a first fluid 130 to control a size of the core-shell particles and shell thickness. This may alternatively, or additionally, include adjusting those parameters of a third fluid. The fluid physical properties could be either adjusted by temperature regime (heating up or cooling down the fluid), or by selecting a fluid with proper properties at room temperature. For example, as pressure of such fluid increases relative to the pressure of the second fluid 140, the thickness of the shell formed by that fluid will decrease.

This may include adjusting 863 the pressure of the second fluid 140 to control a size of the core-shell particles. This may alternatively, or additionally, include adjusting the pressure of a fourth fluid. For example, as pressure of such fluid increase relative to the pressure of the first fluid 130, the volume of the core will increase, and the flow rate of core-shell produced particles will increase too.

In some embodiments, the second tubular member 120, the first lumen 113, and the pressure of the first fluid 130 may be configured to provide a core-shell particle having an outer diameter that is about 200 microns or less. The required size of the generated core-shell particles can be manipulated by combining the surface tension of the first fluid 130, the size of the first lumen 113 controlled either independently or via the pressure of the first fluid 130, the size of the second lumen 114 and the pressure of the second liquid.

In some embodiments, at least 10 mL/min of the core-shell particles may pass through a single second lumen 114, which can be achieved by a selecting an appropriate combination of the pressure in the second tubular member 120, and surface tension, density and viscosity of the first fluid 130.

The disclosed approach can be used as a process technology for producing a product, for example, for the production of aerosols with liquid or solidified core-shell particles carrying a medicine which can be directed into, e.g., human airways (e.g., nasal drug delivery).

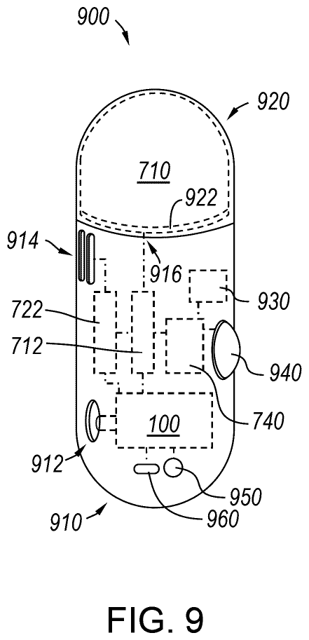

A simplistic example can be seen in FIG. 9. There, a drug delivery device 900 shown having a first housing 910 with a port 912 for dispensing the formed aerosol. The device 100 as disclosed herein is configured to direct particles towards the first port. The device is operably coupled (e.g., via one or more pumps 712, 722) to a source of gas (here, the source is the atmospheric air, through air inlets 914) and to a source of a fluid (shown here as first fluid source 710) for delivery to the user. In some embodiments, a second housing 920 may be removably couplable to the first housing (e.g., via threads, interfacing extrusions and depressions, tabs, etc.). The second housing may include a fluid source containing a active pharmaceutical ingredient (API) or other material for delivery to a user. In some embodiments, the second housing may include, e.g., a film 922, such as a metallized film, configured to be pierced by, e.g., a needle 916 operably coupled to the device 100, such that when the second housing is coupled to the first housing, the film is pierced, and material in the second housing can flow to the device 100. The device may include other necessary components for using the delivery device, such as a battery b coupled to a controller 740. The device may include, e.g., a button 940 for activating the device, and may include one or more other indicators 950 or displays, or ports 960 (such as a charging port or an I/O communications port).

In various aspects, an alternate system may be provided, the system configured to create micron-size droplets, submicron-size droplets, or particles containing microencapsulated materials.

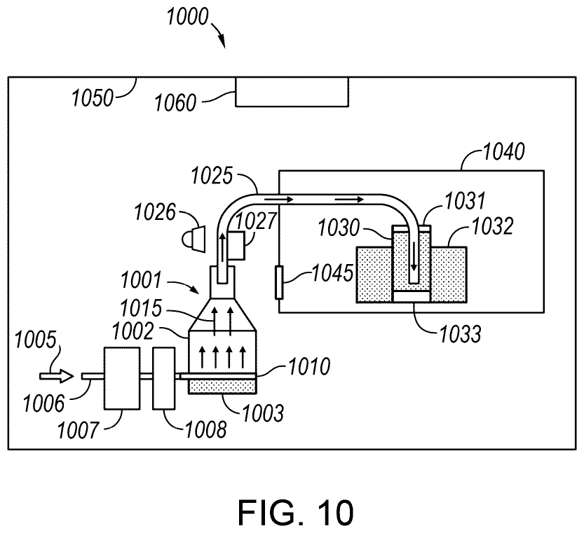

Referring to FIG. 10, a system 1000 may include atomizing device 1001. The chamber may be placed within a chemical fume hood 1050 with a vent 1060. A liquid 1003 forming including a plurality of immiscible liquid layers is in an atomization chamber 1002 within the atomizing device. A source of a gas 1005 is provided to a tube 1010 in the atomization chamber through tubing 1006. The gas feed may optionally be controlled by a regulator 1007, and may optionally include a pressure gauge 1008 downstream of the regulator. The tube 1010 may be configured to be partially submerged in the liquid. The tube may include openings through a sidewall of the tube, the openings arranged such that at least some openings are configured to direct a gas jet towards a bubble on a surface of the liquid to form micron-size droplets, submicron-size droplets, or particles containing microencapsulated materials.

The liquid may include a plurality of immiscible liquid layers.

The plurality of immiscible liquid layers may include a first layer comprising a first material R, and a second layer comprising a second material G, and a third material B in the first layer and/or the second layer, where R, G, and B are selected such that γ_{RB}>γ_{RG}+γ_{GB}, where γ_{RB} is the interface surface tension between the materials R and B, γ_{RG} is the interface surface tension between the materials R and G, and γ_{GB} is the interface surface tension between the materials G and B.

The compressed air produces an aerosol of droplets suspended in air 1015.

The aerosol can then be transported via a hose 1025 (e.g., a “guiding tube”) towards, e.g., a collecting chamber 1040 with a HEPA filter 1045. Thus, the system may include a guiding tube coupled to a top portion of the atomization chamber. The guiding tube may be ultraviolet (UV)-transparent. The guiding tube may be configured to have heated, thermo-insulated or cooled walls. The guiding tube may include a bottom portion coupled to the atomization chamber. The bottom portion and/or sidewalls of the guiding tube may be configured to have apertures for entrainment of outside ambient gas to mix with an aerosol in the guiding tube.

The system may include an ultraviolet (UV) light source 1026 configured to illuminate an aerosol in the guiding tube. The system may include an electrical heating 1027 or cooling coil coupled to the guiding tube.

The system may include a parabolic mirror configured to concentrating solar energy irradiating the guiding tube.

The system may include a pyrolyzer coupled to the guiding tube. In some embodiments, the system may include a burner coupled to an end of the guiding tube, the burner configured to solidify, dehydrate, or pyrolyze aerosol droplets.

The system may include at least one chamber configured to form a dry particle aerosol via solvent evaporation of a submicron droplet aerosol. The system may include a particle collector configured to collect dry particles from a dry particle aerosol. The system may include a liquid, solid or electrostatic filter to capture particulate material from an aerosol stream flowing in the guiding tube.

Inside the collecting chamber the aerosol flow may be directed into a chilled droplet trap including an aluminum cylindrical vessel 1030 with a filter 1031. In some embodiments, the vessel may be at least partially surrounded by a melting water ice bath 1032. The aerosol passing through the chilled droplet trap was cooled down and thus its entropy was reduced, promoting the decrease of aerosol surface area and coalescence of droplets to form bigger ones. Heavier droplets sedimented on the walls of the droplet trap and merged driven by gravity to form a liquid volume 1033 on the bottom of the trap.

The micron-size droplets, submicron-size droplets, or particles containing microencapsulated material may include a single-layer shell. The droplets or particles with a single layer shell may include a single-material core. The droplets or particles with a single layer shell may include a multi-material core. The micron-size droplets, submicron-size droplets, or particles containing microencapsulated material comprises a multi-layer shell. The droplets or particles with a multi-layer shell may include a single-material core. The droplets or particles with a multi-layer shell may include a multi-material core. In some embodiments, all shells may be liquid or solid, or one or more shells may be liquid and one or more shells may be solid. In some embodiments, the core may be liquid or solid, or the core may include a mixture of solid and liquid materials.

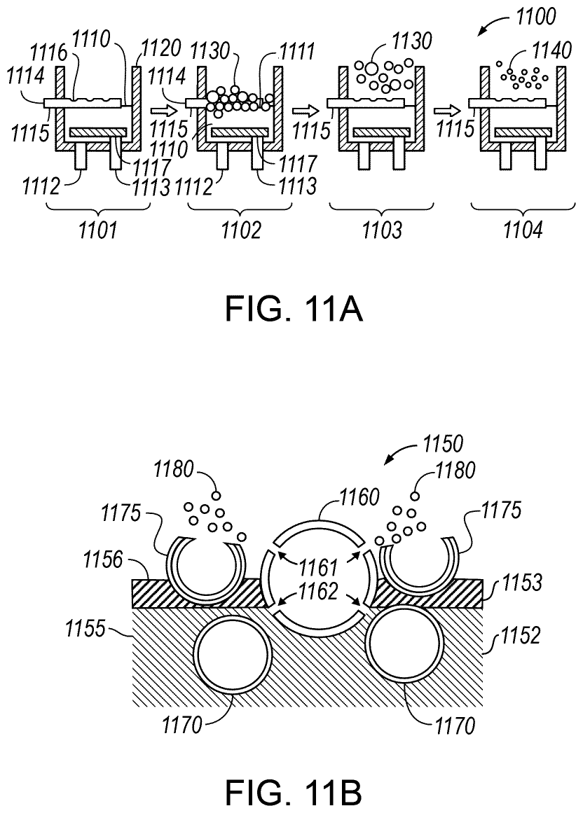

FIGS. 11A-11B show different approaches that can be utilized inside the atomization chamber.

In FIG. 11A, one technique 1100 is shown as generally involving providing the liquid 1110 into a vessel 1120. In preferred embodiments, the vessel is, or forms a portion of, an atomization chamber. The vessel is preferably comprised of glass, stainless steel, and/or any non-reactive material appropriate for containing the specific liquid in use. The vessel will generally be at least partially enclosed. The vessel 1120 may have one or more inlets or ports 1112, 1113, 1114. One inlet 1112 may be configured to allow the liquid to be pumped into the vessel. As will be understood by those of skill in the art, the vessel may contain a sensor (not shown) configured to detect the level of the liquid in the vessel, and a processor (not shown) may be utilized to pump liquid into the vessel if the sensor determines a threshold level of liquid is not present. One inlet 1113 may be configured to allow air to be pumped into the vessel to generate bubbles. As will be understood by those of skill in the art, this inlet may be operably connected to, e.g., a compressed gas storage tank (not shown) via at least one valve or regulator (not shown). And one inlet 1114 may be configured to connect to a perforated tube or pipe 1115, the perforated tube or pipe 1115 configured to have a plurality of holes 1116 through the tube or pipe wall, in order to allow a gas to be directed towards bubbles at or near the surface of the liquid. Preferably, the perforated tube or pipe 1115 is positioned so as to be partially submerged, having at least some holes 1116 above the surface of the liquid and at least some holes at or below the surface of the liquid. In some embodiments, the tube extends across the atomization chamber. In some embodiments, the tube is configured to allow more than one pressurized gas to be connected to it, thereby allowing a mixture of gasses to enter the atomization chamber through the tube. In some embodiments, the inlet 1113 is operably connected to a one-way valve that is configured to allow a connection to the gas supply to be removably attached (e.g., via quick-disconnect fittings). In some embodiments, each end of the tube is operably connected to a one-way valve, and each one-way valve is configured to be removably connected to one or more gas supplies (e.g., one or more compressed gas tanks, etc.)

In a preferred embodiment, no aeration diffuser disc or ring 1117 is utilized. Instead, an arrangement as depicted in FIG. 11B is utilized. In the preferred embodiment 1150, a tube 1160, and more preferably a flexible tube, with openings (e.g., ports, nozzles, perforations or holes) 1161, 1162 through the side walls of the tube is provided. The tube 1160 is positioned within an atomization chamber such that at least some of the openings 1161 are positioned above the air-liquid interface (sometimes referred to as just “the surface”) 1156 of the multi-layer liquid 1155, and at least some of the openings 1162 are positioned below the surface 1156. When a gas is provided to an inlet of the tube 1160, bubbles 1170 are first formed in the bulk liquid from gas exiting the openings 1162 below the surface, initially having just a single shell layer, but additional layers are added to the outer shell as the bubbles pass through each layer 1152, 1153. When the gas exiting through openings 1161 above the surface, they interact disrupt the multi-layered bubbles 1175, forming multi-layered particles 1180.

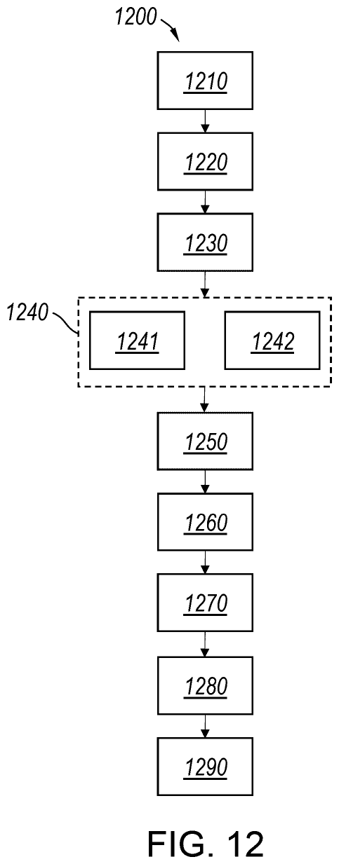

Thus, in various aspects, a method for creating micron-size droplets, submicron-size droplets, or particles containing microencapsulated materials may be provided. Referring to FIG. 12, a method 1200 may include providing 1210 a liquid comprising a plurality of immiscible liquid layers.

The layers may include one or more aqueous layers. The layers may include one or more anhydrous layers. The layers may include a silicone layer.

The method may include aerating 1220 the liquid in an atomization chamber to form bubbles passing through each of the plurality of immiscible liquid layers, such that the bubbles rise to a surface of the liquid. The method may include forming 1230 a submicron droplet aerosol by causing a gas jet to be directed through an opening in a tube towards at least one of the bubbles in the atomization chamber.

The method may include adjusting 1240 temperature in guiding tube coupled to a top portion of the atomization chamber. This may include heating 1241 the aerosol in the tube. This may include cooling 1242 the aerosol in the tube.

The method may include entraining 1250 outside ambient gas through apertures in a portion of the guiding tube coupled to the atomization chamber and/or sidewalls of the guiding tube to mix with an aerosol in the guiding tube.

The method may include photopolymerizing 1260 a material in a droplet or particle by directing ultraviolet (UV) light towards an aerosol in the guiding tube.

The method may include solidifying, dehydrating, and/or pyrolyzing 1270 aerosol droplets.

The method may include forming 1280 a dry particle aerosol via solvent evaporation of the submicron droplet aerosol.

The method may include forming 1290 a powder of submicron or nano-structured particles by passing the dry particle aerosol through a particle collector.

What is claimed is:

1. A device for generating substantially uniform core-shell particles, comprising:
a first tubular member 110 having a first end and a second end, the first tubular member 110 having a first lumen 113 and at least one second lumen 114, where the first lumen 113 extends from the first end to the second end, and where each second lumen 114 extends from an external surface of the first tubular member 110, through a sidewall of the first tubular member 110, to connect to the first lumen 113 at a location a distance in an axial direction from the first end; and
at least one second tubular member 120 having a first end positioned external to the first tubular member 110 and a second end within the first lumen 113 and directed towards one of the at least one second lumen 114, the at least one second tubular member 120 extending through the sidewall of the first tubular member 110.

2. The device of claim 1, further comprising at least one connector, each connector operably coupled to the first end of the first tubular member 110, the second end of the first tubular member 110, or the first end of one of the at least one second tubular member 120.

3. The device of claim 2, wherein the first tubular member 110 comprises an elastic material.

4. The device of claim 3, wherein the at least one second lumen 114 opens or expands when a pressurized liquid is provided into the first lumen 113.

5. The device of claim 4, wherein the at least one second tubular member 120 comprises a rigid material.

6. The device of claim 5, wherein the at least one second tubular member 120 has an inner diameter D that is 10 μm<D<1 mm and a wall thickness T that is 10 μm<T<1 mm.

7. The device of claim 1, wherein the device comprises a single second lumen 114 and a single second tubular member 120.

8. The device of claim 1, wherein the device comprises a plurality of second lumen 114 and a plurality of second tubular members 120, each directed towards one of the plurality of second lumen 114.

9. The device of claim 1, further comprising at least one third tubular member 510 having an inner diameter larger than an outer diameter of the first tubular member 110, the first tubular member 110 and the at least one third tubular member 510 being concentrically positioned, and configured to generate core-shell particles having multiple shells around a core.

10. The device of claim 9, wherein the at least one third tubular member 510 has at least one third lumen 514 that is positioned such that particles exiting the at least one second lumen 114 will also pass through the at least one third lumen 514.

11. The device of claim 1 or 9, further comprising at least one fourth tubular member 620 having an inner diameter larger than an outer diameter of the at least one second tubular member 120, the at least one second tubular member 120 and the at least one fourth tubular member 620 being concentrically positioned, and configured to generate a core comprising multiple materials.

12. A system, comprising:
a device of claim 1;
a first fluid source 710 operably coupled to the first end of the first tubular member 110, the first fluid source 710 configured to provide a first fluid 130; and
a second fluid source 720 operably coupled to the first end of the at least one second tubular member 120, the second fluid source 720 configured to provide a second fluid 140.

13. The system of claim 12, wherein the first fluid 130 is a liquid and the second fluid is a gas.

14. The system of claim 12, wherein the first fluid 130 is a first liquid and the second fluid 140 is a second liquid different from the first liquid.

15. The system of claim 12, wherein the first fluid 130 and second fluid 140 are free of surfactants.

16. The system of claim 12, further comprising a container to collect core-shell particles travelling in a path extending away from the at least one second lumen 114.

17. The system of claim 12, further comprising at least one controller configured to control a flow of fluids through the device to allow a core-shell particle to be formed and directed out of the at least one second lumen 114.

18. The system of claim 17, further comprising a drying means, a photopolymerization means, or a pyrolysis means coupled to the at least one controller and configured to transform at least one layer of the core-shell particle formed by the device from a liquid to a solid.

19. The system of claim 17, wherein the core-shell particle is transformed in an aerosol.

20. The system of claim 17, wherein the core-shell particle is transformed on a surface.

21. A kit, comprising:
at least one device of claim 1; and
a drying means and/or a photopolymerization means.

22. A method for generating substantially uniform layered core-shell particles, comprising:
providing a first fluid 130 to the first lumen 113 of a device of claim 1, a pressure of the first fluid 130 causing the second lumen 114 to open and form a fluid film that spans the open second lumen 114; and
generating substantially uniform core-shell particles by providing a second fluid 140 to the second tubular member 120 configured to direct the second fluid 140 through the fluid film, resulting in a core-shell particles formed having a shell comprising the first fluid 130 surrounding a core comprising the second fluid 140.

23. The method of claim 22, further comprising drying the core-shell particle.

24. The method of claim 22, further comprising photopolymerizing the shell and/or core of the core-shell particle.

25. The method of claim 22, further comprising pyrolyzing the core-shell particle.

26. The method of claim 22, further comprising allowing a chemical reaction to occur in at least one layer of the core-shell particle.

27. The method of claim 22, further comprising collecting the core-shell particle.

28. The method of claim 22, further comprising allowing the core-shell particle to pass through one additional fluid stream passing through at least one tubular member concentrically positioned around the first tubular member 110, creating a multi-shell sphere around a core of gas or liquid.

29. The method of claim 22, further comprising passing at least one additional fluid through at least one additional tubular member concentrically positioned around the second tubular member 120, creating a single-shell sphere with a multiple-material core of gas, liquid, or combination thereof.

30. The method of claim 22, wherein the first fluid 130 and second fluid 140 are free of surfactants.

31. The method of claim 22, wherein each core-shell particles comprises one of:
a microsphere having a one-layer fluid shell and a one-material fluid core;
a microsphere having a multi-layer fluid shell and a one-material fluid core;
a microsphere having a one-layer fluid shell and a multi-material fluid core; or
a microsphere having a multi-layer fluid shell and a multi-material fluid core.

32. The method of claim 31, wherein the fluid shell comprises a liquid.

33. The method of claim 31, wherein the fluid shell comprises a solid.

34. The method of claim 31, wherein the fluid core comprises a gas.

35. The method of claim 31, wherein the fluid core comprises a liquid.

36. The method of claim 31, wherein the fluid core comprises a solid.

37. The method of claim 22, wherein the pressure of the first fluid 130 is adjusted to control an outlet area of the second lumen 114.

38. The method of claim 22, wherein the pressure of the first fluid 130 is adjusted to control a size of the core-shell particles.

39. The method of claim 38, wherein second tubular member 120, the first lumen 113, and the pressure of the first fluid 130 are configured to provide a core-shell particle having an outer diameter that is about 200 microns or less.

40. The method of claim 22, wherein at least 10 mL/min of the core-shell particles pass through a single second lumen 114.

41. The method of claim 22, further comprising allowing the core-shell particles to form a foam.

42. A system for creating micron-size droplets, submicron-size droplets, or particles containing microencapsulated materials, the system comprising:
an atomization chamber; and
a tube within the atomization chamber, the tube configured to be partially submerged in a liquid, the tube comprising openings through a side wall of the tube, the openings arranged such that at least some openings are configured to direct a gas jet towards a bubble on a surface of the liquid to form micron-size droplets, submicron-size droplets, or particles containing microencapsulated materials;
wherein the liquid comprises a plurality of immiscible liquid layers.

43. The system of claim 42, wherein plurality of immiscible liquid layers includes a first layer comprising a first material R, and a second layer comprising a second material G, and a third material B in the first layer and/or the second layer, where R, G, and B are selected such that γ_{RB}>γ_{RG}+γ_{GB}, where γ_{RB} is the interface surface tension between the materials R and B, γ_{RG} is the interface surface tension between the materials R and G, and γ_{GB} is the interface surface tension between the materials G and B.

44. The system of claim 42, wherein the micron-size droplets, submicron-size droplets, or particles containing microencapsulated material comprises a single-layer shell.

45. The system of claim 44, wherein the micron-size droplets, submicron-size droplets, or particles containing microencapsulated material comprises a single-material core.

46. The system of claim 44, wherein the micron-size droplets, submicron-size droplets, or particles containing microencapsulated material comprises a multi-material core.

47. The system of claim 42, wherein the micron-size droplets, submicron-size droplets, or particles containing microencapsulated material comprises a multi-layer shell.

48. The system of claim 47, wherein the micron-size droplets, submicron-size droplets, or particles containing microencapsulated material comprises a single-material core.

49. The system of claim 47, wherein the micron-size droplets, submicron-size droplets, or particles containing microencapsulated material comprises a multi-material core.

50. The system of claim 49, wherein all shells are liquid, all shells are solid, or one or more shells are liquid and one or more shells are solid.

51. The system of claim 50, wherein the core is liquid, the core is solid, or the core a mixture of solid and liquid materials.

52. The system of claim 42, further comprising a guiding tube coupled to a top portion of the atomization chamber.

53. The system of claim 52, wherein the guiding tube is ultraviolet (UV)-transparent.

54. The system of claim 52, wherein the guiding tube is configured to have heated, thermo-insulated or cooled walls.

55. The system of claim 52, wherein the guiding tube includes a bottom portion coupled to the atomization chamber, the bottom portion and/or sidewalls of the guiding tube configured to have apertures for entrainment of outside ambient gas to mix with an aerosol in the guiding tube.

56. The system of claim 52, further comprising:
an ultraviolet (UV) light source configured to illuminate an aerosol in the guiding tube;
an electrical heating or cooling coil coupled to the guiding tube;
a parabolic mirror configured to concentrating solar energy irradiating the guiding tube;
a burner coupled to an end of the guiding tube, the burner configured to solidify, dehydrate, or pyrolyze aerosol droplets;
at least one chamber configured to form a dry particle aerosol via solvent evaporation of a submicron droplet aerosol;
a particle collector configured to collect dry particles from a dry particle aerosol; and/or
a liquid, solid or electrostatic filter to capture particulate material from an aerosol stream flowing in the guiding tube.

57. A method for creating micron-size droplets, submicron-size droplets, or particles containing microencapsulated materials, the method comprising:
providing a liquid comprising a plurality of immiscible liquid layers;
aerating the liquid in an atomization chamber to form bubbles passing through each of the plurality of immiscible liquid layers, such that the bubbles rise to a surface of the liquid; and
forming a submicron droplet aerosol by causing a gas jet to be directed through an opening in a tube towards at least one of the bubbles in the atomization chamber.

58. The method of claim 57, wherein plurality of immiscible liquid layers includes a first layer comprising a first material R, and a second layer comprising a second material G, and a third material B in the first layer and/or the second layer, where R, G, and B are selected such that γ_{RB}>γ_{RG}+γ_{GB}, where γ_{RB} is the interface surface tension between the materials R and B, γ_{RG} is the interface surface tension between the materials R and G, and γ_{GB} is the interface surface tension between the materials G and B.

59. The method of claim 57, wherein the micron-size droplets, submicron-size droplets, or particles containing microencapsulated material comprises a single-layer shell.

60. The method of claim 59, wherein the micron-size droplets, submicron-size droplets, or particles containing microencapsulated material comprises a single-material core.

61. The method of claim 59, wherein the micron-size droplets, submicron-size droplets, or particles containing microencapsulated material comprises a multi-material core.

62. The method of claim 57, wherein the micron-size droplets, submicron-size droplets, or particles containing microencapsulated material comprises a multi-layer shell.

63. The method of claim 62, wherein the micron-size droplets, submicron-size droplets, or particles containing microencapsulated material comprises a single-material core.

64. The method of claim 62, wherein the micron-size droplets, submicron-size droplets, or particles containing microencapsulated material comprises a multi-material core.

65. The method of claims 64, wherein all shells are liquid, all shells are solid, or one or more shells are liquid and one or more shells are solid.

66. The method of claim 65, wherein the core is liquid, the core is solid, or the core a mixture of solid and liquid materials.

67. The method of claim 57, further comprising:
heating or cooling a guiding tube coupled to a top portion of the atomization chamber;
entraining outside ambient gas through apertures in a portion of the guiding tube coupled to the atomization chamber and/or sidewalls of the guiding tube to mix with an aerosol in the guiding tube;
photopolymerizing a material in a bubble by directing ultraviolet (UV) light towards an aerosol in the guiding tube;
solidifying, dehydrating, or pyrolyzing aerosol droplets;
forming a dry particle aerosol via solvent evaporation of the submicron droplet aerosol; and/or
optionally, forming a powder of submicron or nano-structured particles by passing the dry particle aerosol through a particle collector.
